(19) United States
(12) Patent Application Publication
(10) Pub. No.: US 2012/0309767 A1
(43) Pub. Date: Dec. 6, 2012

(54) PROCESS FOR THE PREPARATION OF ALPHA FORM OF IMATINIB MESYLATE

(76) Inventors: Sharma Ashwani, Navi Mumbai (IN); Madhav Hire Chandrabhan, Navi Mumbai (IN); Rohit Chaturvedi, Navi Mumbai (IN); Rajan Gupte, Navi Mumbai (IN)

(21) Appl. No.: 13/578,918

(22) PCT Filed: Feb. 15, 2011
(86) PCT No.: PCT/IN11/00097

§ 371 Date: Aug. 14, 2012

(30) Foreign Application Priority Data
Feb. 15, 2010 (IN) 398/MUM/2010

(51) Int. Cl. A61K 31/506; A61P 35/00; C07D 401/14

(52) U.S. Cl. 514/252.18; 544/295

(57) ABSTRACT

The present invention provides an improved process for the preparation of alpha form of imatinib mesylate with (long needle) and α-crystal form (small needle) in a consistent manner and novel alpha crystal forms of imatinib mesylate. The present invention in particular provides a reproducible and efficient process. In particular the present invention provides a efficient process which gives higher yields and consistent results.

### CROSS-REFERENCE TO RELATED PATENT APPLICATIONS

The present application claims benefit of the filing date of Indian Provisional Patent Application No. 398/MUM/2010 filed on Feb. 15, 2010, which is entirely incorporated herein by reference.

### TECHNICAL FIELD

The present invention relates to an improved process for the preparation of alpha form imatinib mesylate and novel alpha crystal forms of imatinib mesylate. The present invention in particular provides a reproducible and efficient process resulting in high yields of alpha form without compromising on the purity of the alpha form.

### BACKGROUND ART

Imatinib is chemically known as 4-(4-methylpiperazin-1-ylmethyl)-N-[4-methyl-3-(4-pyridin-3-yl)pyrimidin-2-ylamino)phenyl]benzamide of the formula (I). The compound Imatinib mesylate is sold under the brand name GLIVEC® for the treatment of Chronic Myeloid Leukemia (CML) and gastrointestinal stromal tumors (GIST). It has also been approved for the treatment of patients with kit [CD 117] positive unresectable and recently it has been approved for the treatment of pediatric patients with Philadelphia chromosome positive (Ph+) chronic myeloid leukemia in chronic phase.

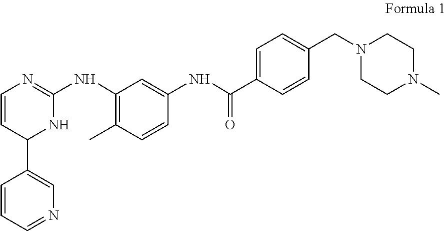

The preparation of Imatinib and its use, especially as an anti-tumor agent, are described in EP-A-0564409. The compound is exemplified in these publications only in free from (not as a salt). Imatinib Mesylate, the alpha and the beta crystal form thereof, as well as its pharmaceutical use are described in U.S. Pat. No. 6,894,051.

Another polymorph of Imatinib mesylate, the so called H-1 form, is described in WO2004/106326. Two new crystalline polymorphic Form I and Form II of Imatinib mesylate and their use are described in WO 2006/054 314. Delta and epsilon crystal form of Imatinib mesylate and there processes for the preparation and pharmaceutical composition are reported in WO 2007/023182. Later on, it has been reported in WO2007/059963 that under certain conditions new crystalline form of methanesulphonate salt are prepared as F, G, H, I and K crystal form.

Various patents such as WO 99/03854, WO2006/0223816, WO 2005/095379, WO 2006/048890 reports the process for the preparation of α-crystal form with needle shaped crystal. Another new polymorphic form named α₂ is disclosed in Indian patent application No. 706/CHE/04 and pharmaceutical composition containing it.

It is reported in WO 99/03854, the process for the preparation of Alpha form by suspending imatinib base in ethanol, methane sulphonic acid is added and heated the reaction mixture under reflux for 20 minute and then filtered clear at 65° C. The filtrate was evaporated down to 50% and the residue filtered off at 25° C. (filter material A). The mother liquor was evaporated to dryness. The residue and the filter material A were suspended in ethanol dissolved under reflux with the addition of water. Cooling overnight at 25° C., filtration and drying yielded Imatinib mesylate α-crystal form.

The above process for the preparation of α-crystal form suffers from the following disadvantages in that it is not reproducible and inventors after conducting experiments have found that the process resulted in beta form, which proves that the patent is not enabled to give a consistent results.

WO 2005/095379 describes a method for preparing α-crystal form by using 0.95-0.99 molar ration of methane sulphonic acid per mole of imatinib, in the reaction mixture. The method described in the '379 application generally includes addition of methane sulphonic acid to a solution of imatinib in an alcohol or a mixture of alcohol and ester, cooling and seeding at temperature close to the temperature of crystallization (i.e., after completing the addition of methane sulphonic acid and after cooling), and further cooling and filtering. However, this process does not have industrial viability.

WO2006/024863 also describes a method of preparing crystalline imatinib mesylate α-form; however, the '863 application teaches micronizing the product order to change the undesirable crystalline needle form and obtain desirable form of solid.

WO2006/0223816 describes a process for the preparation of α-form from organic solvents with imatinib and methane sulphonic acid dissolved therein, and seed crystals of imatinib mesylate in substantially pure α-form, wherein the seed crystals are added before imatinib mesylate begins to precipitate from the solution. The above process also describes that the seeding is to be carried out before the addition of methanesulfonic acid or at the beginning of the acid addition phase, but sufficiently in advance of the time that solid imatinib mesylate begins precipitating from solution.

WO 2006/048890 explains that imatinib mesylate is dissolved in Methanol:water (1:4) ratio at 25-30° C. to get the clear solution. The solution was concentrated in agitated thin film drier (ATFD) with flow rate of 18 to 20 lit per hour using Peristaltic pump at vapor temperature of about 50-55° C. under vacuum for 60-90 minutes. The product obtained is α-crystalline form of Imatinib mesylate.

The previously known method for producing the alpha-crystal form of methane sulfonic acid addition salt of the compound of formula (I) involves the precipitation of the salt from its solution in non-alcoholic solvents. It is a known fact that alpha-crystal form obtained by the conventional processes were inconsistent resulting in unstable crystal alpha form which was found to be hygroscopic in nature and having flow characteristics unsuited for pharmaceutical preparations.

Imatinib is a drug used to treat certain types of cancer. It is used in treating chronic myelogenous leukemia (CML), gastrointestinal stromal tumors (GISTs) and other cancers. It is the first member of a new class of agents that act by specifically inhibiting a certain enzyme that is characteristic of a particular cancer cell, rather than non-specifically inhibiting and killing all rapidly dividing cells.

Imatinib is often cited as an example of pharmaceutical industry innovation, hence looking to the need of the hour, the inventors of the present invention has successfully developed an improved process to prepare α-crystal form with (long needle) and α-crystal form with (small needle) in a consistent and reproducible manner.

### STATEMENT OF THE INVENTION

The present invention provides an improved process for the preparation of alpha form imatinib mesylate and novel alpha crystal forms of imatinib mesylate. The present invention in particular provides a reproducible and efficient process resulting in high yields of alpha form without compromising on the purity of the alpha form. This process is simple and industrially viable thereby providing stable alpha form of imatinib mesylate with (long needle) and α-crystal form (small needle) in a consistent mariner.

### OBJECTIVE OF THE INVENTION

It is the object of the current invention to provide an improved process for the preparation of alpha form imatinib mesylate with (long needle) and α-crystal form (small needle) in a consistent manner and novel alpha crystal forms of imatinib mesylate.

It is the object of the current invention to provide a reproducible and efficient process resulting in high yields of alpha form without compromising on the purity of the alpha form of imatinib mesylate

It is the object of the present invention to provide stable formulation of the same.

### SUMMARY OF INVENTION

The present invention relates to a crystalline form of methanesulfonicacid addition salt of 4-(4-methylpiperazin-1-ylmethyl)-N-[4-methyl-3-(4-pyridin-3-yl)pyrimidin-2ylamino)phenyl]benzamide (imatinb mesylate). More specifically, the invention relates to the reproducible and efficient process resulting in high yields of stable alpha crystalline form without compromising on the purity of the form of imatinib mesylate

In one aspect of the invention, the present invention provides process for preparation of alpha form of imatinib mesylate from imatinib base. In one preferred embodiment the present invention employs imatinib base which is at least 99% pure. In one particular embodiment the present invention uses imatinib base which has been purified inhouse or can be commercially obtained.

Imatinib base may be prepared according to processes in prior art as for example in EP 0564409.

According to one aspect of the present invention there is provided a simple and industrial process for the preparation of alpha crystalline form of the imatinib mesylate with (long needle) and α-crystal form (small needle) in a consistent manner.

In one aspect the present invention has studied the effect of various solvents, moisture limit, temperature and amount of methanesulfonic acid to prepare the small needles and long needles forms of alpha crystalline form.

In one aspect, the present invention has studied the stability of these forms.

### BRIEF DESCRIPTION OF DRAWINGS

The following drawings form part of the present specification and are included to further demonstrate certain aspects of the present disclosure, the inventions of which can be better understood by reference to one or more of these drawings in combination with the detailed description of specific embodiments presented herein.

FIG. 1 shows the X-ray diffraction diagram of the alpha-crystal form (long needle) of imatinib mesylate

FIG. 2: shows the X-ray diffraction diagram of the alpha-crystal form (small needle) of imatinib mesylate

FIG. 3: shows the DSC of long needles

FIG. 4: shows the DSC of small needles

FIG. 5: shows the FTIR of long needles

FIG. 6: shows the FTIR of small needles

FIG. 7: shows the microscopic picture of the alpha crystal form (long needles) of the imatinib mesylate

FIG. 8: shows the microscopic picture of the alpha crystal form (small needles) of the imatinib mesylate

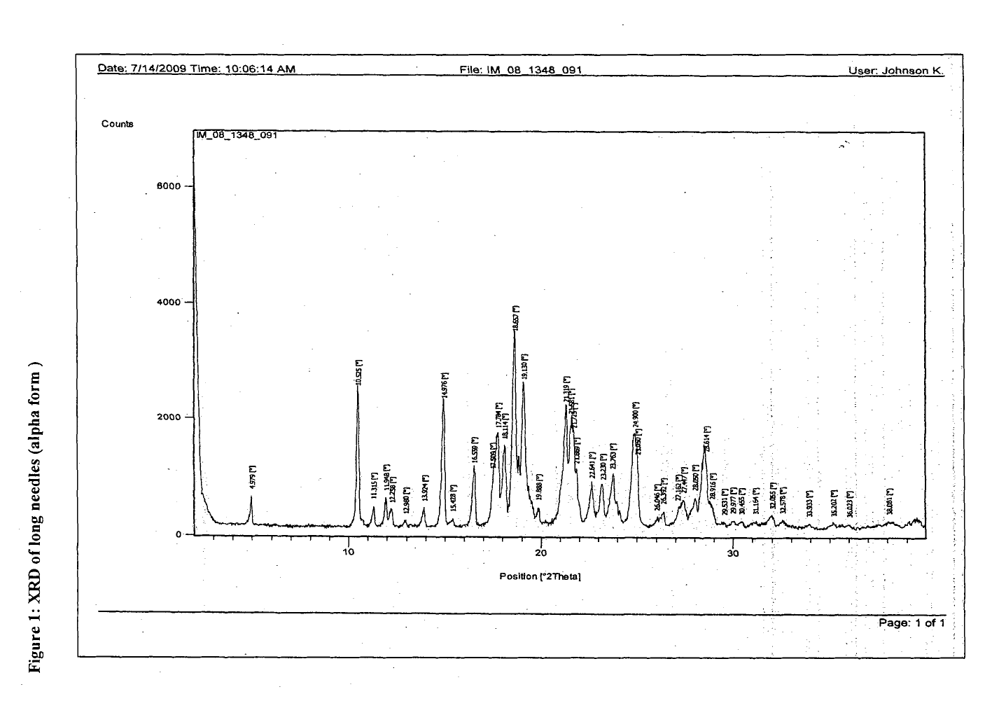

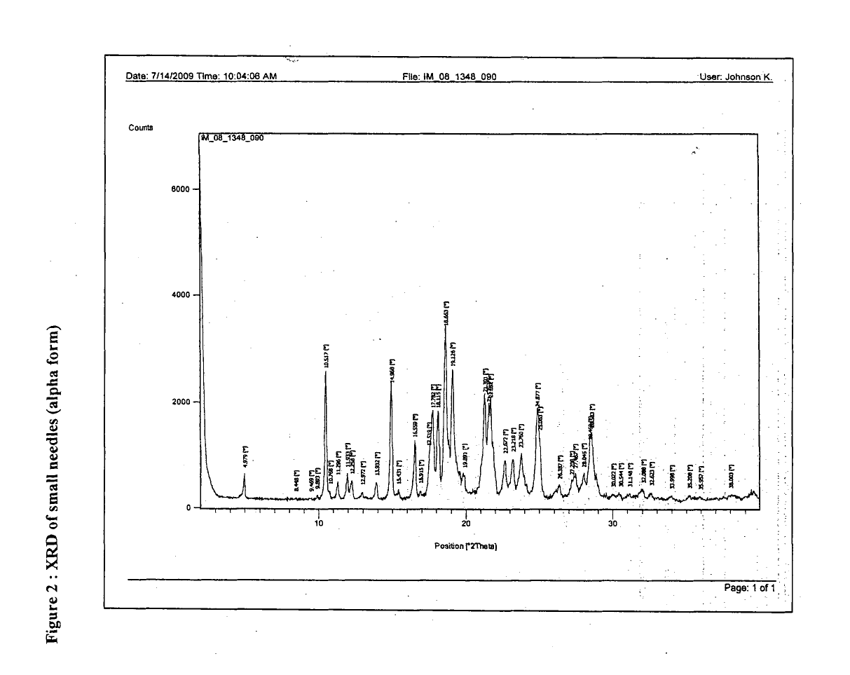

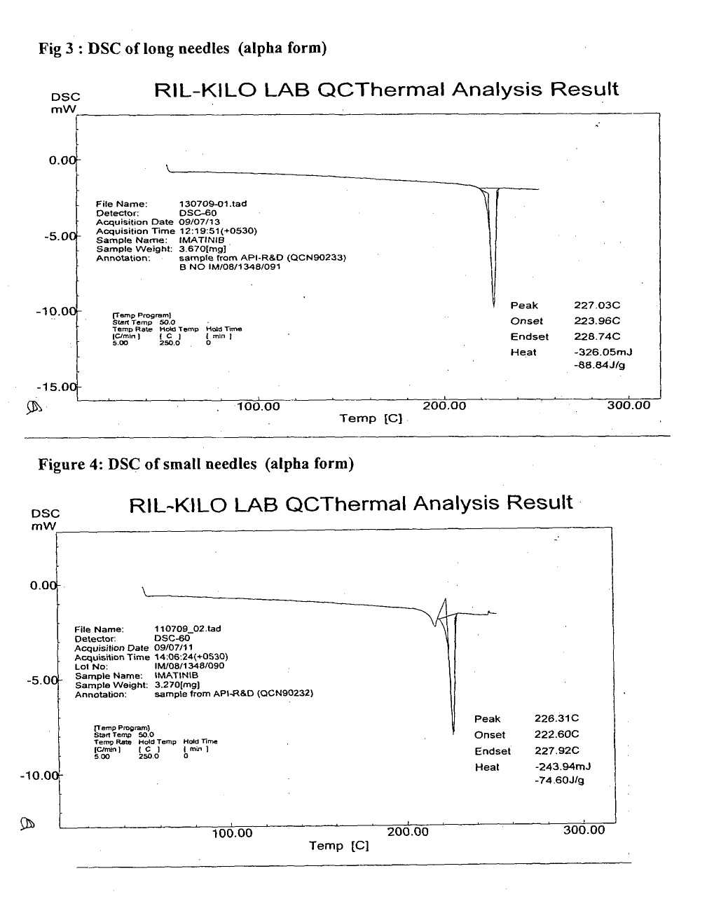

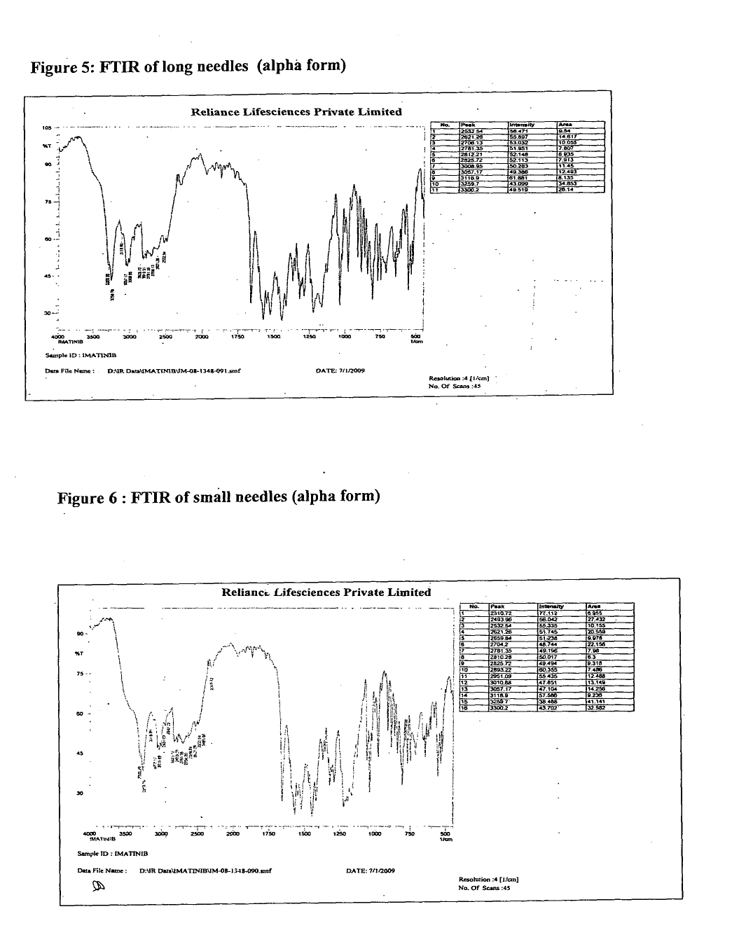

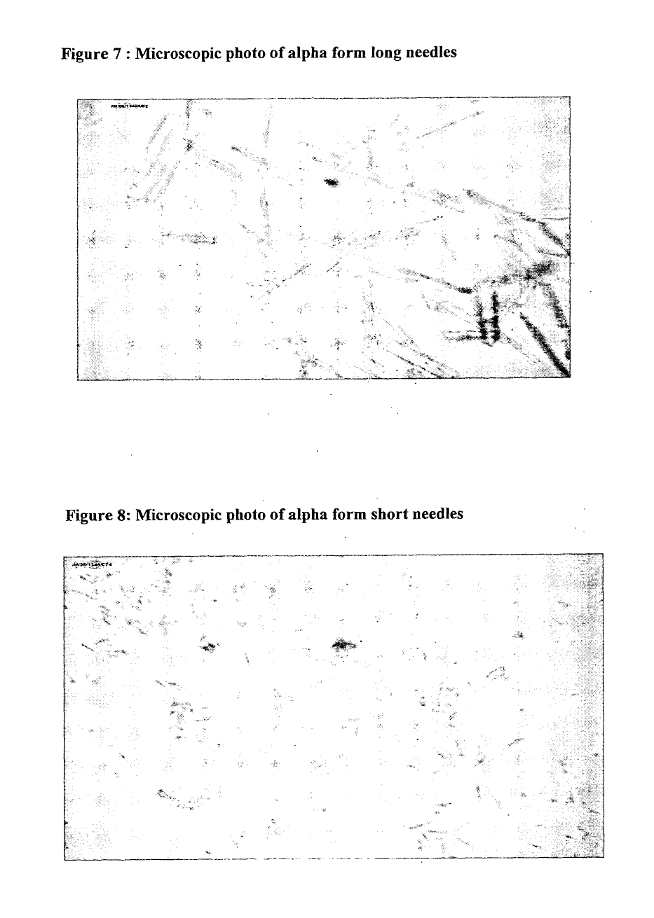

### DESCRIPTION OF EMBODIMENTS

### Definitions

The term “imatinib” as used herein refers to the base form of 4-(4-methylpiperazin-1-ylmethyl)-N-[4-methyl-3-(4-pyridin-3-yl)pyrimidin-2ylamino)phenyl]benzamide.

The term “imatinib mesylate” as used herein refers the mesylate salt of imatinib.

### Preferred Embodiments and the Scope

The present invention provides a simple and efficient process for imatinib mesylate alpha form in a consistent manner and novel alpha crystal forms of imatinib mesylate.

The present invention provides a process comprising suspending the imatinib base in a solvent preferably isopropanol and adding a solution of methane sulfonic acid in the same solvent to favor alpha form. Further the reaction is maintained at a higher temperature for the completion of the salt form and subsequently cooled to room temperature. The precipitate thus obtained was then filtered and dried at a higher temperature.

The present invention has studied that the purity of the imatinib base is also critical for the formation of the alpha crystal form. In one preferred embodiment the purity of the base of at least 99%, favor the formation of alpha crystals in a consistent manner in a pure form.

The present invention has studied that the choice of solvent is critical for the formation of alpha form. It was observed that alcoholic solvents such as isopropanol (IPA), t-butanol and ketonic solvents such as MEK favored alpha form. Preferably the present invention utilizes IPA for the formation of alpha form of imatinib mesylate.

Further the moisture content in the solvent was also studied. It was noted that the moisture content of around 1% was critical for the formation of alpha crystal. Along with moisture content in the solvent, it was critically noted that choice of the molar ratio of the base to methane sulfonic acid favored alpha crystal form.

In one preferred embodiment the anhydrous solvent used in the process of the present invention has a moisture content of 0.01 to 1.5%. Generally it is preferred for the moisture content to be at least 0.1%, more preferably at least 0.2%, more preferably still at least 0.5%. It is also generally preferred for the moisture content to be no more than 1.2%. Typically it is around 1%, e.g. 0.8 to 1.2%.

In one preferred embodiment of the invention, if the moisture content of the solvent is less than 0.1% then the methanesulphonic acid is brought into contact with the imatinib base, in the solvent, at a contact temperature of above 30° C. (preferably 35-70° C., more preferably 40-60° C., e.g. 40-45° C. or 55-60° C.), and/or the reaction mixture, i.e. the mixture of solvent, imatinib base and methanesulphonic acid, is warmed to and kept at a temperature of at least 30° C. (preferably 40-60° C., more preferably 45-50° C.) for at least 10 minutes (preferably 20-40 minutes, typically around 30 minutes) after the reaction mixture is made up. Obviously this warming step is used only to increase the temperature of the mixture, and so if the contact temperature is above 30° C. and is followed by a warming step, then the warming step will increase the temperature to a higher temperature, e.g. 40-60° C.

In this aspect of the invention, if small needles are desired, then it is preferred for the contact temperature to be less than 50° C., such as 35-50° C., more preferably 40-45° C., and/or for the mixture to be heated to 40-55° C. after being made up, more preferably 45-50° C. Typically both a raised contact temperature and a subsequent warming step are used. Alternatively, if long needles are desired, then it is preferred for the contact temperature to be above 50° C., such as 50-65° C., more preferably 55-60° C. Generally a subsequent warming step is not needed if long needles are being made.

It is also generally preferred to reflux the mixture after the reaction mixture has been made up and (if relevant) the warming step has taken place. Preferably the reflux step lasts for at least 10 minutes, typically at least 30 minutes, preferably 1-4 hours, and usually around 2 hours.

The technique for extracting the crystalline product from the reaction mixture is not specifically limited. If an elevated contact has been used and/or a warming and/or reflux step carried out, then it is preferred for the reaction mixture then to be cooled (or left to cool) down to room temperature, e.g. 20-25° C. A filter step is preferably used for extraction (after cooling, if relevant), followed by drying at e.g. 50-150° C., preferably 80-120° C., more preferably 80-100° C., typically 95-100° C. Drying normally lasts for 10-30 hours, typically 15-20 hours. Preferably a washing step is carried out after the filter step but before drying. The same solvent as was used in the reaction mixture is typically used for washing.

It was observed that less than 1 mole of methansulfonic acid with respect to base did not favor the formation of the alpha form.

It is preferred that at least as much methanesulfonic acid as base is used in the process of the present invention. Preferably the ratio of methanesulfonic acid to base is X:1 wherein X is greater than 1, preferably 1.2 to 10, more preferably 1.5 to 5 and typically around 2.

It is preferred that the weight ratio of base to methanesulfonic acid is Y:1 wherein Y is 2 to 10, preferably 3 to 7 and typically around 5.

It was found that addition of methanesulfonic acid at a temperature range of 30-60° C. favored alpha form with high purity with respect to residual solvent limits and gave high yields.

Surprisingly it was also noted while studying the effect of the addition temperature of methane sulfonic acid that the alpha form precipitated as small and long needles at different temperatures. It was observed that addition below 50 deg C. favored small needles and above 50 deg C. favored long needles of the alpha form.

On obtaining the above the results of different forms the inventors has characterized the two forms of alpha form and the stability data has been detailed in the foregoing experiments and results.

The main features of the product of the present invention

a) alpha form with residual solvent content within the acceptable limits
b) alpha form which is stable
c) alpha form in two shapes: small needle and long needles.

The present invention provides alpha crystals of imatinib mesylate which
(i) have one of their three most intense peaks (preferably their most intense peak) in the region of 18.6 to 18.7 degrees 2θ when subjected to XRPD; and/or
(ii) have one of their three most intense peaks (preferably their second most intense peak) in the region of 10.5 to 10.6 degrees 2θ when subjected to XRPD; and/or
(iii) have one of their three most intense peaks (preferably their third most intense peak) in the region of 19.1 to 19.2 degrees 2θ when subjected to XRPD; and/or
(iv) melt in the region of 224-229° C. when subjected to DSC (in this context, melting generally does not occur outside that temperature range).

In a preferred aspect the alpha crystals have one, preferably two, and more preferably three of their most intense peaks as measured by XRPD located in one, two or all of the following three ranges: 10.5 to 10.6, 18.6 to 18.7, and 19.1 to 19.2 degrees 2θ.

Further preferred features of the alpha crystals as determined by XRPD are as follows. Preferably the alpha crystals have a peak at 5.0 degrees 2θ±0.1 (preferably±0.05). Preferably the alpha crystals have a peak at 10.5 degrees 2θ±0.1 (preferably±0.05). Preferably the alpha crystals have a peak at 11.3 degrees 2θ±0.1 (preferably±0.05). Preferably the alpha crystals have a peak at 11.9 degrees 2θ±0.1 (preferably±0.05). Preferably the alpha crystals have a peak at 12.3 degrees 2θ±0.1 (preferably±0.05). Preferably the alpha crystals have a peak at 13.9 degrees 2θ±0.1 (preferably±0.05). Preferably the alpha crystals have a peak at 15.0 degrees 2θ±0.1 (preferably±0.05). Preferably the alpha crystals have a peak at 16.6 degrees 2θ±0.1 (preferably±0.05). Preferably the alpha crystals have a peak at 17.5 degrees 2θ±0.1 (preferably±0.05). Preferably the alpha crystals have a peak at 17.8 degrees 2θ±0.1 (preferably±0.05). Preferably the alpha crystals have a peak at 18.1 degrees 2θ±0.1 (preferably±0.05). Preferably the alpha crystals have a peak at 18.7 degrees 2θ±0.1 (preferably±0.05). Preferably the alpha crystals have a peak at 19.1 degrees 2θ±0.1 (preferably±0.05). Preferably the alpha crystals have a peak at 19.9 degrees 2θ±0.1 (preferably±0.05). Preferably the alpha crystals have a peak at 21.3 degrees 2θ±0.1 (preferably±0.05). Preferably the alpha crystals have a peak at 21.6 degrees 2θ±0.1 (preferably±0.05). Preferably the alpha crystals have a peak at 21.7 degrees 2θ±0.1 (preferably±0.05). Preferably the alpha crystals have a peak at 22.7 degrees 2θ±0.1 (preferably±0.05). Preferably the alpha crystals have a peak at 23.2 degrees 2θ±0.1 (preferably±0.05). Preferably the alpha crystals have a peak at 218 degrees 2θ±0.1 (preferably±0.05). Preferably the alpha crystals have a peak at 24.9 degrees 2θ±0.1 (preferably±0.05). Preferably the alpha crystals have a peak at 25.1 degrees 2θ±0.1 (preferably±0.05). Preferably the alpha crystals have a peak at 27.2 degrees 2θ±0.1 (preferably±0.05). Preferably the alpha crystals have a peak at 27.5 degrees 2θ±0.1 (preferably±0.05). Preferably the alpha crystals have a peak at 28.0 degrees 2θ±0.1 (preferably±0.05). Preferably the alpha crystals have a peak at 28.6 degrees 2θ±0.1 (preferably±0.05). Preferably the alpha crystals have a peak at 32.0 degrees 2θ±0.1 (preferably±0.05). Most preferred crystals have all of the peaks set out above.

The alpha form of the crystals of the present invention is a stable alpha form. Preferably, the alpha crystals of the invention have a water content (as measured by KF) of not more than 1% w/w, preferably not more than 0.8% w/w, more preferably not more than 0.7% w/w, and typically less than 0.6% w/w. As shown by the data in table 4 below, the alpha crystals of the invention are able to retain this low water content even after storage for 3 months at 40° C. and a relative humidity (RH) of 75. Thus, preferably the alpha crystals of the invention retain a water content of not more than 1% w/w, preferably not more than 0.8% w/w, more preferably not more than 0.7% w/w, and typically less than 0.65% w/w after being stored at 1, 2 or even 3 months at 40° C. and a relative humidity (RH) of 75. This is an example of how the alpha form of the crystals of the present invention is a stable alpha form.

Preferably, the alpha crystals of the invention have not more than 1.0% w/w total impurities, preferably not more than 0.5% w/w, more preferably not more than 0.2% w/w, more preferably still not more than 0.1% w/w and typically not more than 0.05% w/w. As shown by the data in table 4 below, the alpha crystals of the invention are able to retain this low impurity content even after storage for 3 months at 40° C. and a relative humidity (RH) of 75. Thus, preferably the alpha crystals of the invention retain an impurity content of not more than 1.0% w/w total impurities, preferably not more than 0.5% w/w, more preferably not more than 0.2% w/w, more preferably still not more than 0.1% w/w and typically not more than 0.05% w/w after being stored at 1, 2 or even 3 months at 40° C. and a relative humidity (RH) of 75. This is an example of how the alpha form of the crystals of the present invention is a stable alpha form. In this context the impurities are typically measured by HPLC, e.g. as described in the Examples below.

Preferably, the alpha form of the present invention is at least 99.9% w/w pure, preferably at least 99.95% pure, as measured by HPLC (e.g. as described in the Examples below). As shown by the data in table 4 below, the alpha crystals of the invention are able to retain a very high purity even after storage for 3 months at 40° C. and a relative humidity (RH) of 75. Thus, preferably the alpha crystals of the invention retain a purity level of at least 99.0% w/w pure, preferably at least 99.5% w/w, typically at least 99.7% w/w, after being stored at 1, 2 or even 3 months at 40° C. and a relative humidity (RH) of 75. This is an example of how the alpha form of the crystals of the present invention is a stable alpha form.

In one preferred embodiment the present invention provides a substantially pure form of alpha crystals of imatinib mesylate characterized by one or more of the preferred characteristic features described herein.

In another preferred embodiment the present invention provides a crystalline form consisting essentially of alpha crystals of imatinib mesylate characterized by one or more of the preferred characteristic features described herein.

The main features of the process of the present invention is

a) the critical moisture content of the solvent which should be about 1%
b) the critical molar ratio of the base to the methanesulfonic acid of at least 1:1
c) the critical temperature of addition of methane sulfonic acid at a range of 30-60° C.
d) the drying temperature of the product at 80-100° C.

It is submitted that the above features has resulted in a process giving consistent and stable alpha form which is exemplified in the examples below:

In this process it is noted that alpha crystal form of imatinb mesylate obtained by the process of present invention involves addition of methane sulphonic acid, refluxing, cooling and isolation of desired crystal by filtration.

The process for the present invention can be conveniently carried out in alcoholic or ketonic solvents. In a preferred aspect of the invention the solvent is an alcohol wherein the hydrocarbyl group is a straight or branched aliphatic group with 2 to 6 carbon atoms. The alcoholic solvents may be selected from C₂ to C₄ alcohols, preferably ethanol or tert-butanol and most preferably isopropanol. Typically the alcohol has 3 or 4 carbon atoms. In another aspect, it is preferred that the solvent is a ketone containing straight or branched aliphatic hydrocarbyl groups, such that the ketone has a total of 3 to 6 carbon atoms, preferably 3 to 5 carbon atoms, and typically 4 or 5 carbon atoms, such as methyl ethyl ketone (MEK).

There is no particular method reported in the literature for the preparation of α-crystal form with (long needle) and α-crystal form with (small needle). The inventors have developed the process for the preparation of α-crystal form with (long needle) and α-crystal form with (small needle).

Further the inventors have critically and experimentally studied the various parameters which plays important role in forming the desired crystal structure like, choice of solvent, moisture limit, temperature and ratio of methane sulphonic acid to synthesize the α-crystal form with (long needle) and α-crystal form with (small needle) in a consistent and reproducible manners refers examples.
i) It has been observed that temperature plays an important part in determining the physical nature of the crystal form as observed under microscope.
ii) Addition of methane sulphonic acid at higher temperature (55-60° C.) results in formation of α-crystal form (long needles)
iii) Addition of methane sulphonic acid at 30-45° C. results in formation of α-crystal form (small needles)
iv) Another important factor controlling the formation of alpha form is moisture. It has been observed that moisture in IPA lower than 1.0% leads to formation of alpha form with (small as well as long needles)
v) The molar ratio of methane sulphonic acid is another factor responsible for crystal formation. Molar ratio lower than 1 mole leads to absence of alpha forms

The inventors of the present invention has observed that the residual solvent levels by following the process mentioned in the patent nos. WO2005/077933 A1 and WO2006/024863 A1 are not as per ICH guidelines even after drying at elevated temperature at 100° C. The present invention has provided a process with which we have solved the residual solvent problem in the final product as per ICH guidelines. As explained above and demonstrated by the data presented in tables 1 to 4 below, the alpha crystals of imatinib mesylate of the present invention are new forms of imatinib mesylate, which have good stability characteristics. The low solvent levels in the crystals of the invention and also their good stability characteristics, makes them well suited for use in pharmaceutical formulations, as they can retain the required crystal structure during the processing required to prepare a pharmaceutical product. Thus, in a preferred embodiment the present invention provides a process of preparing a pharmaceutical composition, which process comprises
(a) preparing alpha crystals of imatinib mesylate according to any one of claims 1 to 13; and
(b) combining the alpha crystals with a pharmaceutically acceptable carrier or diluent.

The process of present invention is very suitable for industrial application, particularly as a pharmaceutical. The imatinib mesylate according to the invention is suitable for use in therapy. It is useful in methods of treating cancer, particularly leukemia (most commonly chromic myeloid leukemia) and GIST. It is also useful in manufacture of medicament for treating such diseases.

The preparation of alpha crystal form is exemplified by the following examples:

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The preparation of alpha crystal form is exemplified by the following examples:

### Example 1

### Preparation of α-form (small needle) of 4-(4-methylpiperazin-1-ylmethyl)-N-[4-methyl-3-(4-pyridin-3-yl)pyrimidin-2ylamino)phenyl]benzamide methanesulphonate

Imatinib base (10 gms) was suspended in 140 ml of isopropanol. Methanesulfonic acid (1.985 gms) in 10 ml anhydrous Isopropanol (moisture 0.09%) added slowly during 5 minutes at 40-45° C. and raised temperature at 45-48° C. and maintained for 30 minute. The reaction mass was heated to reflux at 80-85° C. for 2 hours and slowly cooled to 20-25° C. during 35-40 minutes. Filtered at 20-25° C. and washed with 30 ml isopropanol. The wet cake was dried at 95-100° C. for 16-20 hours. The yield was 11.64 gms (97.48%). The residual Isopropyl alcohol content was 3100 ppm.

### Example 2

### Preparation of α-form (long needle) of 4-(4-methylpiperazin-1-ylmethyl)-N-[4-methyl-3-(4-pyridin-3-yl)pyrimidin-2ylamino)phenyl]benzamide methanesulphonate

Imatinib base (10 gms) was suspended in 140 ml of isopropanol methanesulfonic acid (1.985 gms) in 10 ml anhydrous isopropanol added slowly during 5 minutes at 55-60° C. The reaction mass was heated to reflux at 80-85° C. for 2 hours and slowly cooled to 20-25° C. during 35-40 minutes. Filtered at 20-25° C. and washed with 30 ml isopropanol. The wet cake was dried at 95-100° C. for 16-20 hours. The yield was 11.9 gms (99.6%) α-form. The residual Isopropyl alcohol content was 3300 ppm.

### Example 3

### Preparation of 4-(4-methylpiperazin-1-ylmethyl)-N-[4-methyl-3-(4-pyridin-3-yl)pyrimidin-2ylamino)phenyl]benzamide methanesulphonate in Isopropyl alcohol

Imatinib base (5 gms) was suspended in 60 ml of isopropanol. Methanesulfonic acid (0.992 gms) in 15 ml isopropanol (moisture=1.1%) added slowly during 15-20 minutes at 25-30° C. The reaction mass was heated to reflux at 80-85° C. for 2 hours and slowly cooled to 20-25° C. during 35-40 minutes. Filtered at 20-25° C. and washed with 25 ml isopropanol. The wet cake was dried at 95-100° C. for 16-20 hours. The yield was 5.6 gms (94.4%) α-form.

### Example 4

### (Reference Example) Preparation of 4-(4-methylpiperazin-1-ylmethyl)-N-[4-methyl-3-(4-pyridin-3-yl)pyrimidin-2ylamino)phenyl]benzamide methanesulphonate in Isopropyl alcohol

Imatinib base (10 gms) was suspended in 140 ml of isopropanol methanesulfonic acid (1.985 gms) in 10 ml isopropanol (moisture=1.78%) added slowly during 5 minutes at 25-30° C. The reaction mass was heated to reflux at 80-85° C. for 2 hours and slowly cooled to 20-25° C. during 35-40 minutes. Filtered at 20-25° C. and washed with 30 ml isopropanol. The wet cake was dried at 95-100° C. for 16-20 hours. The yield was 11.59 gms (97.1%). The XRD data obtained did not match with that of α-form.

### Example 5

### (Reference Example) Preparation of 4-(4-methylpiperazin-1-ylmethyl)-N-[4-methyl-3-(4-pyridin-3-yl)pyrimidin-2ylamino)phenyl]benzamide methanesulphonate in Isopropyl alcohol

Imatinib base (5 gms) was suspended in 60 ml of isopropanol. Methanesulfonic acid (0.992 gms) in 15 ml isopropanol (moisture=2.0%) added slowly during 15-20 minutes at 55-60° C. The reaction mass was heated to reflux at 80-85° C. for 2 hours and slowly cooled to 20-25° C. during 35-40 minutes. Filtered at 20-25° C. and washed with 30 ml isopropanol. The wet cake was dried at 95-100° C. for 16-20 hours. The yield was 5.8 gms (97.1%). The XRD data obtained did not match with that of α-form.

### Example 6

### Preparation of 4-(4-methylpiperazin-1-ylmethyl)-N-[4-methyl-3-(4-pyridin-3-yl)pyrimidin-2ylamino)phenyl]benzamide methanesulphonate in Isopropyl alcohol

Imatinib base (5 gms) was suspended in 60 ml of isopropanol. Methanesulfonic acid (1.07 gms) in 15 ml anhydrous isopropanol (moisture=0.21%) added slowly during 15-20 minutes at 25-30° C. The reaction mass was heated to reflux at 80-85° C. for 2 hours and slowly cooled to 20-25° C. during 35-40 minutes. Filtered at 20-25° C. and washed with 25 ml isopropanol. The wet cake was dried at 95-100° C. for 16-20 hours. The yield was 5.7 gms (95.4%). The XRD data obtained match with that of α-form.

### Example 7

### (Reference Example) Preparation of 4-(4-methylpiperazin-1-ylmethyl)-N-[4-methyl-3-(4-pyridin-3-yl)pyrimidin-2ylamino)phenyl]benzamide methanesulphonate in Isopropyl alcohol

Imatinib base (5 gms) was suspended in 60 ml of isopropanol. Methanesulfonic acid (0.953 gms) in 15 ml anhydrous isopropanol (moisture=0.09%) added slowly during 15-20 minutes at 25-30° C. The reaction mass was heated to reflux at 80-85° C. for 2 hours and slowly cooled to 20-25° C. during 35-40 minutes. Filtered at 20-25° C. and washed with 25 ml isopropanol. The wet cake was dried at 95-100° C. for 16-20 hours. The yield was 5.87 gms (98.3%) The XRD data obtained did not match with that of α-form.

### Example 8

### Preparation of 4-(4-methylpiperazin-1-ylmethyl)-N-[4-methyl-3-(4-pyridin-3-yl)pyrimidin-2ylamino)phenyl]benzamide methanesulphonate in. Isopropyl alcohol

Imatinib base (10 gms) was suspended in 120 ml of isopropanol. Methanesulfonic acid (1.985 gms) in 30 ml anhydrous isopropanol (moisture=0.15%) added slowly during 15-20 minutes at 18-20° C. The reaction mass was heated to reflux at 80-85° C. for 2 hours and slowly cooled to 20-25° C. during 35-40 minutes. Filtered at 20-25° C. and washed with 50 ml isopropanol. The wet cake was dried at 95-100° C. for 16-20 hours. The yield was 11.6 gms (97.1%) α-form.

### Example 9

### Preparation of a form (small needle) of 4-(4-methylpiperazin-1-ylmethyl)-N-[4-methyl-3-(4-pyridin-3-yl)pyrimidin-2ylamino)phenyl]benzamide methanesulphonate

Imatinib base (5 gms) was suspended in 60 ml of tert-butanol. Methanesulfonic acid (0.993 gms) in 15 ml tert-butanol added slowly during 20 minutes at 35-37° C. The reaction mass was heated to reflux at 80-85° C. for 2 and half hours and reaction mass cooled gradually to 20-25° C. during 40-45 minutes. Filtered at 20-25° C. and washed with 25 ml tert-butanol. The wet cake was dried under vacuum at 80° C. for 6 hours. The yield was 5.4 gms (91.5%) α-form.

### Example 10

### Preparation of a form (small needle) of 4-(4-methylpiperazin-1-ylmethyl)-N-[4-methyl-3-(4-pyridin-3-yl)pyrimidin-2ylamino)phenyl]benzamide methanesulphonate

Imatinib base (5 gms) was suspended in 60 ml of methyl ethyl ketone. Methanesulfonic acid (0.993 gms) in 15 ml methyl ethyl ketone added slowly during 20 minutes at 35-37° C. The reaction mass was heated to 80-85° C. for 2.5 hours and reaction mass cooled gradually to 20-25° C. during 40-45 minutes. Filtered at 20-25° C. and washed with 20 ml methyl ethyl ketone. The wet cake was dried under vacuum at 65° C. for 6 hours. The yield was 5.2 gms (87.1%) α-form.

### Analysis of the Polymorphs Obtained in the Present Invention:

The following instruments and conditions were used for the analysis of the polymorph whose results are detailed in a tabulated form later in the specification:
1. DSC: Melting point is determined by means of DSC thermogram using a SHIMADZU DSC-60. DSC (“differential scanning calorimetry”)

| Condition: | [Temp program] |
|---|---|
| Start temp | 50.0 |

| Temp Rate | Hold temp | Hold time |
|---|---|---|
| [C./min] | [C.] | [min] |
| 5.00 | 250.0 | 0 |

2. FTIR (Fourier transform infrared spectrophotometer)
   Make: SHIMADZU, IR Prestige-21
   Sample preparation: KBr pellets
3. Microscope: For microscopy Olympus microscope with 40× lens
4. HPLC: Used Agilent 1200, VWD detector, system gradient Instrument conditions are:

CHROMATOGRAPHIC SYSTEM:

| | | |
|---|---|---|
| 1 | Mobile phase | A:B (Gradient) |
| 2 | Buffer | 7.5 g 1-Octane sulfonic acid sodium salt in |
| | | 100 ml of water adjusted to pH 3.0 with ortho |
| | | phosphoric acid. |
| 3 | Column | Symmetry C18, 150 × 4.6, 3.5 μ |
| 4 | Wavelength | 240 nm |
| 5 | Flow rate | 1.0 ml/min |
| 6 | Column temperature | 27° C. |
| 7 | Diluent | Water:Methanol (45:55 v/v) |
| 8 | Run time | 65.0 min |
| 9 | Injection volume | 20.0 μl |

Gradient Program

| Time (Min) | % Mobile phase A | % Mobile phase B |
|---|---|---|
| 0.0 | 100 | 0 |
| 15.0 | 80 | 20 |
| 45.0 | 35 | 65 |
| 55.0 | 35 | 65 |
| 60.0 | 100 | 0 |
| 65.0 | 100 | 0 |

5. XRPD:

XRD diffraction was performed on X-Ray powder diffractometer. P analytical X'pert Pro powder diffractometer. Cu-tube scanning parameters: CuKα radiation, λ=1.54060 Å Continuous scan at a rate of 0.0170° 2 theta/51.0404 sec. Start position 2.0084° 2 theta and end position 39.9884° 2 theta

TABLE 1
XRPD characteristics of Imatinib mesylate α-form (long needles) of
example 2

| Angle 2 Theta | d value Angstrom | Intensity % |
|---|---|---|
| 4.979 | 17.734 | 15.25 |
| 10.525 | 8.398 | 71.48 |
| 11.314 | 7.814 | 10.14 |
| 11.947 | 7.401 | 14.71 |
| 12.258 | 7.214 | 8.34 |
| 13.924 | 6.354 | 9.08 |
| 14.976 | 5.910 | 64.43 |
| 16.558 | 5.349 | 28.94 |
| 17.509 | 5.061 | 25.75 |
| 17.784 | 4.983 | 47.47 |
| 18.114 | 4.893 | 41.54 |
| 18.657 | 4.752 | 100.00 |
| 19.129 | 4.635 | 73.57 |
| 19.888 | 4.460 | 8.59 |
| 21.318 | 4.164 | 61.39 |
| 21.580 | 4.114 | 54.81 |
| 21.725 | 4.087 | 47.07 |
| 21.869 | 4.060 | 28.01 |
| 22.641 | 3.924 | 20.42 |
| 23.230 | 3.825 | 19.55 |
| 23.762 | 3.741 | 25.25 |
| 24.900 | 3.572 | 48.07 |
| 25.050 | 3.551 | 33.15 |
| 27.162 | 3.280 | 9.02 |
| 27.447 | 3.246 | 12.95 |
| 28.049 | 3.178 | 14.40 |
| 28.614 | 3.117 | 35.21 |
| 28.916 | 3.085 | 10.19 |
| 32.055 | 2.789 | 5.71 |

TABLE 2
XRPD characteristics of Imatinib mesylate α-form (small needles) of
example 1

| Angle 2 Theta | d value Angstrom | Intensity % |
|---|---|---|
| 4.979 | 17.733 | 14.44 |
| 10.516 | 8.405 | 76.06 |
| 11.295 | 7.826 | 10.48 |
| 11.933 | 7.410 | 15.06 |
| 12.258 | 7.214 | 10.92 |
| 13.931 | 6.351 | 9.92 |
| 14.967 | 5.914 | 66.74 |
| 16.559 | 5.349 | 32.54 |
| 17.519 | 5.058 | 26.66 |
| 17.781 | 4.984 | 49.94 |
| 18.115 | 4.892 | 49.94 |
| 18.663 | 4.750 | 100.00 |
| 19.126 | 4.636 | 75.28 |
| 19.891 | 4.459 | 12.87 |
| 21.301 | 4.167 | 59.30 |
| 21.575 | 4.115 | 53.39 |
| 21.691 | 4.093 | 55.88 |
| 22.671 | 3.918 | 22.04 |
| 23.21 | 3.827 | 22.89 |
| 23.760 | 3.741 | 25.63 |
| 24.877 | 3.576 | 50.93 |
| 25.062 | 3.550 | 36.52 |
| 26.384 | 3.374 | 7.21 |
| 27.207 | 3.274 | 9.91 |
| 27.466 | 3.244 | 14.20 |
| 28.040 | 3.179 | 14.59 |
| 28.468 | 3.132 | 31.47 |
| 28.583 | 3.120 | 38.96 |
| 32.088 | 2.787 | 5.78 |

TABLE 3
Comparative XRPD, DSC and IR data of example 2

| | XRPD | XRPD | | | | |
|---|---|---|---|---|---|---|
| | 2θ (deg) | 2θ (deg) | DSC ° C. (±0.2°) | | IR value cm⁻¹ | |
| | α- form | α-form | α- form | α-form | α- form | α-form |
| S no. | (reported) | Observed | (reported) | (Observed) | (reported) | (Observed) |
| 1 | 4.87 | 4.97 | 221-228 | 223.9-228.74 | 3261 | 3259.7-3261 |
| 2 | 10.4 | 10.52 | | | | |
| 3 | 14.85 | 11.31 | | | | |
| 4 | 16.44 | 11.94 | | | | |
| 6 | 17.62 | 14.97 | | | | |
| 7 | 18.04 | 16.55 | | | | |
| 8 | 18.56 | 17.50 | | | | |
| 9 | 19.01 | 17.78 | | | | |
| 10 | 19.78 | 18.11 | | | | |
| 11 | 21.20 | 18.65 | | | | |
| 12 | 21.48 | 19.12 | | | | |
| 13 | 21.62 | 21.31 | | | | |
| 14 | 22.40 | 21.58 | | | | |
| 15 | 22.57 | 21.72 | | | | |
| 16 | 23.09 | 21.86 | | | | |
| 17 | 23.69 | 22.64 | | | | |
| 18 | 24.82 | 23.23 | | | | |
| 19 | 26.23 | 23.76 | | | | |
| 20 | 27.19 | 24.90 | | | | |
| 21 | 27.84 | 25.05 | | | | |
| 22 | 27.96 | 27.44 | | | | |
| 23 | 28.46 | 28.04 | | | | |
| 24 | | 28.61 | | | | |
| 25 | | 28.91 | | | | |

### Stability Study Data of Imatinib Mesylate of Example 2

A stability study was performed on the alpha form of imatinib mesylate at 40° C. and 75 RH for 3 months. The study showed that the alpha imatinib mesylate produced in accordance with the present invention is quite stable as indicated in Table ‘5’ wherein there is no increase in the level of impurities before and after stability study at 40° C. and 75 RH for 3 months and further study is in progress.

TABLE 4

| | Testing | | Results | | | |
|---|---|---|---|---|---|---|
| S. No | parameter | Acceptance criteria | Initial | 1M | 2M | 3M |
| 1 | Descriptions | An off white to pale | White | White | White | White |
| | | yellow crystalline | crystalline | crystalline | crystalline | crystalline |
| | | powder | powder | powder | powder | powder |
| 2 | Identification | IR spectrum of Test | concordant | concordant | concordant | concordant |
| | by IR | sample should | | | | |
| | | concordant with | | | | |
| | | spectrum of working | | | | |
| | | standard of Imatinib | | | | |
| | | Mesylate (Form- α) | | | | |
| 3 | Water content | NMT 1.0% w/w | 0.52 | 0.55 | 0.61 | 0.56 |
| | by KF | | | | | |
| 4 | Related Substance [by HPLC] | | | | | |
| | a) Known impurities | | | | | |
| | 1. Impurity-1 | NMT 0.15% w/w | ND | ND | ND | 0.02 |
| | 2. Impurity-2 | NMT 0.15% w/w | ND | ND | ND | ND |
| | 3. Impurity-3 | NMT 0.15% w/w | ND | ND | ND | ND |
| | b) Unknown | NMT 0.10% w/w | 0.04 | ND | 0.03 | 0.02 |
| | impurity | | | | | |
| | RRT = 0.81 | | | | | |
| | c) Total | NMT 1.0% w/w | 0.04 | ND | ND | ND |
| | impurities | | | | | |
| 5 | Assay [by | Between 98.05% to | 99.98 | 99.94 | 99.70 | 99.75 |
| | HPLC] (on | 102.0%/w (on | | | | |
| | anhydrous) | anhydrous) | | | | |

| | | | | |
|---|---|---|---|---|
| Remarks: | — | Stable | Stable | Stable |

*ND = Not detected
Below 0.02% w/w impurities disregarded.

Solubility data of the alpha form

| S no. | Solvent | α-polymorph |
|---|---|---|
| 1 | Water | 1 gms/2 ml |
| 2 | DMSO | 1 gms/10 ml |
| 3 | Methanol | 1 gms/20 ml |

All of the compositions and methods disclosed and claimed herein can be made and executed without undue experimentation, in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention as defined by the appended claims.

1. A process for the preparation of alpha crystals of imatinib mesylate by reacting imatinib base with methanesulphonic acid in anhydrous solvent.

2. The process according to claim 1, wherein the molar ratio of imatinib base to methanesulphonic acid is at least 1:1.

3. The process according to claim 1, wherein the base is at least 99% pure.

4. The process according to claim 1, wherein the solvent is selected from alcoholic and ketonic solvents.

5. The process according to claim 4, wherein the alcoholic solvent is isopropanol or tert-butanol.

6. The process according to claim 4, wherein the ketonic solvent is methyl ethyl ketone.

7. The process according to claim 1, wherein the anhydrous solvent is having moisture content of 0.01 to 1.5%, and preferably about 1%.

8. The process according to claim 1, wherein the steps of preparation of the alpha crystals of imatinib mesylate comprises:
a) mixing of imatinib base in anhydrous solvent;
b) addition of methanesulfonic acid at a temperature between 30-60° C.;
c) heating the reaction mixture to reflux;
d) cooling and filtering the crystals formed; and
e) drying the crystals at 80-100° C.

9. The process according to claim 1, wherein long needles of alpha crystal of imatinib mesylate are formed when methanesulfonic acid is added between 55-60° C.

10. The process according to claim 1, wherein small needles of alpha crystal of imatinib mesylate are formed when methanesulfonic acid is added between 30-45° C.

11. The process according to claim 1, wherein the alpha form is a stable form.

12. The process according to claim 1, wherein the alpha form is at least 99.9% pure.

13. The process according to claim 1, wherein if the moisture content of the solvent is less than 0.1% then the methanesulphonic acid is brought into contact with the imatinib base, in the solvent, at a contact temperature of above 30° C.

14. A process of preparing a pharmaceutical composition, which process comprises:
a) preparing alpha crystals of imatinib mesylate according to claim 1; and
b) combining the alpha crystals with a pharmaceutically acceptable carrier or diluent.

15. Alpha crystals of imatinib mesylate obtained or obtainable by a process as defined in claim 1.

16. Alpha crystals of imatinib mesylate which
(i) have one of their three most intense peaks in the region of 18.6 to 18.7 degrees 2θ when subjected to XRPD; and/or
(ii) have one of their three most intense peaks in the region of 10.5 to 10.6 degrees 2θ when subjected to XRPD; and/or
(iii) have one of their three most intense peaks in the region of 19.1 to 19.2 degrees 2θ when subjected to XRPD; and/or
(iv) melt in the region of 224-229° C. when subjected to DSC.

17. A method of treating a human or animal body by therapy which comprises administering alpha crystals of imatinib mesylate as defined in claim 15.

18. A method according to claim 17 which is for the treatment of cancer.

19. Alpha crystals of imatinib mesylate as defined in claim 15 for use in the treatment of cancer.

20. Use of alpha crystals of imatinib mesylate as defined in claim 15 in the manufacture of a medicament for use in treating cancer.
